(19) United States
(12) Patent Application Publication
(10) Pub. No.: US 2016/0051219 A1
(43) Pub. Date: Feb. 25, 2016

(54) EVALUATION METHOD FOR RADIOGRAPHING APPARATUS AND PHANTOM USED IN EVALUATION

(71) Applicant: CANON KABUSHIKI KAISHA, Tokyo (JP)

(72) Inventors: Tetsuo Shimada, Hachioji-shi (JP); Osamu Tsujii, Kawasaki-shi (JP)

(21) Appl. No.: 14/825,833

(22) Filed: Aug. 13, 2015

(30) Foreign Application Priority Data
Aug. 20, 2014 (JP) 2014-167821

(51) Int. Cl. A61B 6/00

(52) U.S. Cl. CPC A61B 6/583; A61B 6/502

(57) ABSTRACT

An evaluation method for a radiographing apparatus according to the present invention includes an imaging step of imaging a plurality of base phantoms, each of the base phantoms corresponding to a different imaging system and combined with a common evaluation region, and an evaluation step of performing evaluation among the different imaging systems based on an image in which the common evaluation region has been imaged in the imaging step.

### BACKGROUND OF THE INVENTION

1. Field of the Invention

The present invention relates to an evaluation method for a radiographing apparatus and a phantom used in evaluation of the radiographing apparatus.

2. Description of the Related Art

In an imaging apparatus using radiation such as full-body computed tomography (CT) imaging, breast specific CT imaging, tomosynthesis imaging, and two-dimensional imaging by radiation, the apparatus is evaluated and calibrated by using an evaluating phantom according to each imaging system. In evaluation of a radiographing apparatus, calibration of lesion image extraction performance is performed based on an image in which the evaluating phantom is imaged.

Japanese Patent Application Laid-Open No. 2013-81770 discloses a phantom (X-ray calibration device) modelling an abdominal region of a patient used by a CT imaging apparatus. It also discloses that the phantom is used in checking and evaluating of performance of algorithm for estimating visceral adipose tissue (VAT).

Japanese Patent Application Laid-Open No. 2013-81770 describes an evaluation method using the phantom specialized for the CT imaging apparatus, which is not applicable to other imaging apparatuses (imaging systems). Accordingly, between imaging apparatuses using different imaging systems, it has been difficult to compare and evaluate lesion image extraction performance thereof using the same phantom. As a result, selection of an imaging apparatus (imaging system) according to a target lesion is not easily performed.

### SUMMARY OF THE INVENTION

Accordingly, one or more aspects of the present invention provide an evaluation method for a radiographing apparatus that makes it possible to select an imaging system suitable for a target evaluation region among a plurality of imaging systems.

An evaluation method for a radiographing apparatus according to the present invention includes an imaging step of imaging a plurality of base phantoms, each of the base phantoms corresponding to a different imaging system and combined with a common evaluation region, and an evaluation step of performing evaluation among the different imaging systems based on an image in which the common evaluation region has been imaged in the imaging step.

Further features of the present invention will become apparent from the following description of exemplary embodiments (with reference to the attached drawings).

### BRIEF DESCRIPTION OF THE DRAWINGS

FIG. 1 is a flowchart illustrating an evaluation method for a radiographing apparatus according to a first embodiment.

FIG. 2 is a schematic view illustrating an exemplary base phantom suitable for CT imaging.

FIGS. 3A and 3B are schematic views illustrating an exemplary base phantom for the radiographing apparatus and a tomosynthesis apparatus for a breast.

FIG. 4 is a schematic view illustrating an exemplary base phantom suitable for a breast specific CT apparatus.

FIGS. 5A, 5B, and 5C are schematic views illustrating an exemplary evaluating phantom suitable for evaluating low contrast resolution.

FIGS. 6A and 6B are schematic views illustrating an exemplary evaluating phantom suitable for evaluating high contrast resolution.

FIGS. 7A and 7B are schematic views illustrating an exemplary evaluating phantom suitable for spicule resolution.

FIG. 8 is a schematic view illustrating an exemplary connecting phantom.

### DESCRIPTION OF THE EMBODIMENTS

### First Embodiment

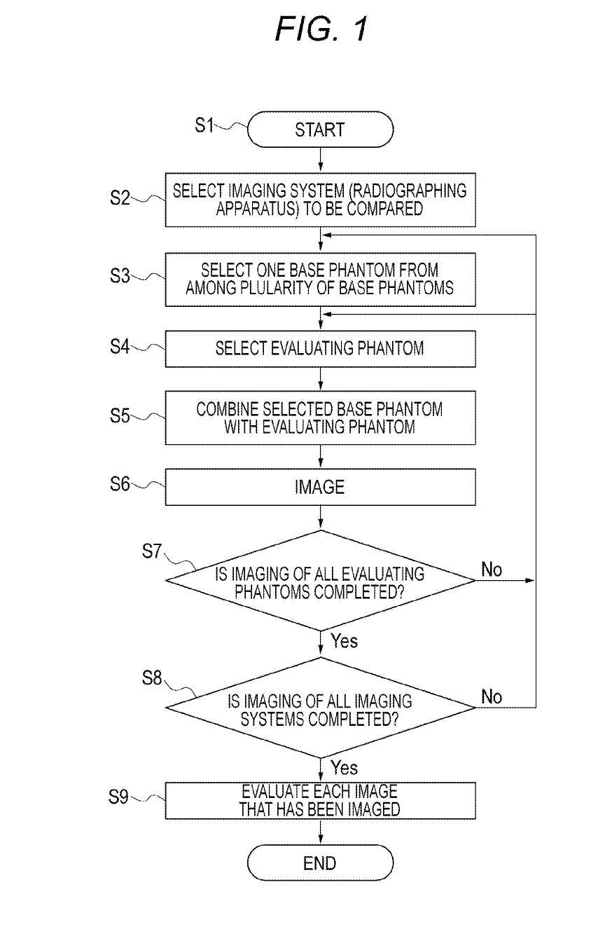

First, an evaluation method for a radiographing apparatus according to a first embodiment is described using FIG. 1. In this embodiment, the evaluation method in which a breast is an object to be imaged is described. Note that the object to be imaged is not limited to the breast; the evaluation method may be applicable by modifying this embodiment as appropriate to any region that may be an object to be imaged by a plurality of imaging systems.

The evaluation method according to this embodiment includes at least an imaging step and an evaluation step. In the imaging step, a plurality of base phantoms, each of the base phantoms corresponding to a different imaging system and combined with a common evaluation region, is imaged. In the evaluation step, the evaluation region that is combined with the base phantom is imaged, and the selected imaging system is evaluated based on an image that has been imaged. Here, a “phantom” refers to a test body used in calibration and measurement related to image quality performance of the radiographing apparatus. The test body has an index for evaluating a predetermined imaging performance. An exemplary phantom includes a test body having a plurality of x-ray transmission portions, each having a radiation transmittance different from each other, and a test body designed to have a substantially uniform radiation transmittance as a whole. The “base phantom” is a phantom that has a shape corresponding to each of the radiographing apparatuses or the imaging systems and that is capable of holding the evaluation region in an attachable and detachable manner. In this embodiment, the evaluation region is configured to be attachable to and detachable from the base phantom. A configuration of the phantom, however, is not limited to this. The evaluation region may also be combined with the base phantom in a state that is not attachable to or detachable from the base phantom. The “evaluation region” is a region having a structure for evaluating imaging performance of each of the radiographing apparatuses or each of the imaging systems. An “evaluating phantom” is a phantom having the evaluation region for evaluating the imaging performance of each of the radiographing apparatuses or each of the imaging systems. Note that a specific configuration of the base phantom and the evaluating phantom is described below.

In step S1 of FIG. 1, a flow of the evaluation method is started. In step S2, the imaging system and the radiographing apparatus to be compared and evaluated are selected. Here, the imaging system refers to a system for obtaining an image of a specimen, and includes at least one imaging system among full-body CT imaging, breast specific CT imaging, tomosynthesis imaging, and two-dimensional imaging. The two-dimensional imaging includes imaging of two-dimensional images such as a still image and a moving image (moving image for fluoroscopy and the like). The imaging system and the radiographing apparatus may be in a one-to-one relationship, or one radiographing apparatus may have a plurality of imaging systems. An imaging system may also include various other imaging systems, whereby it is not to be limited to this.

In step S3, from among a plurality of base phantoms, one base phantom corresponding to at least one of the imaging systems is selected. Here, the base phantom has a shape corresponding to at least one of the imaging systems among the CT imaging, the tomosynthesis imaging, and the two-dimensional imaging. Accordingly, by selecting the base phantom appropriate for the imaging system, it is possible to perform the imaging of the phantom corresponding to each of the imaging systems. Appropriate evaluation can be performed even in a case where a method of holding the phantom is different between the radiographing apparatuses. In a case where the plurality of base phantoms is selected, the imaging is performed by combining each of the base phantoms to the evaluating phantom that is common to the plurality of base phantoms that has been selected.

In step S4, the evaluating phantom that is suitable for extracting a lesion to be a comparison object is selected. As the evaluating phantom, for example, a phantom that determines low contrast resolution evaluation, high contrast resolution evaluation, and spicule resolution evaluation can be used. Detail of each of the base phantoms and each of the evaluating phantoms is described below. Content that can be evaluated in this step is not limited to this, and it is also possible to further use a phantom that evaluates a contrast to noise ratio (CNR) and the like.

In step S5, one base phantom selected in step S3 is combined with the evaluating phantom selected in step S4.

In step S6, the evaluating phantom, which has been combined with the base phantom, is imaged. In step S7, it is determined whether all of the evaluating phantoms that are the objects in step S4 have been imaged. In a case where there is any evaluating phantom that has not been imaged yet, processing returns to step S4, and the imaging is repeated.

In step S8, it is determined whether all of the evaluating phantoms have been imaged by an imaging system and the radiographing apparatus that are objects in step S2. In a case where there is any imaging system or radiographing apparatus by which the evaluating phantoms have not been imaged yet, the processing returns to step S3, and the imaging is repeated.

In step S9, based on an image obtained as a result of imaging the evaluating phantom, which is combined with one base phantom selected, performance evaluation among the imaging systems and among the radiographing apparatuses as well as evaluation of lesion image extraction performance are performed. Furthermore, based on a result of evaluating the image of the evaluating phantom that has been radiographed, it is also possible to set an imaging condition for each of the radiographing apparatuses. Also, by this evaluation method, it is possible to compare intensity and radiation quality of radiation to be irradiated for providing substantially equal lesion image extraction performance among the imaging systems. By this evaluation method, it is possible to determine the intensity and the radiation quality of the radiation. Here, the “substantially equal lesion image extraction performance” means that detection performance of the evaluation region of the evaluating phantom, described below, is the same. Accordingly, an examinee, an engineer, and the like can select an imaging system with a small radiation dosage or an imaging system with short imaging time from among different imaging systems.

By the above evaluation method, in a radiographic examination using the plurality of imaging systems, it is possible to perform the performance evaluation among the imaging systems and among the apparatuses as well as the evaluation of the lesion image extraction performance by using a uniform index (evaluation region).

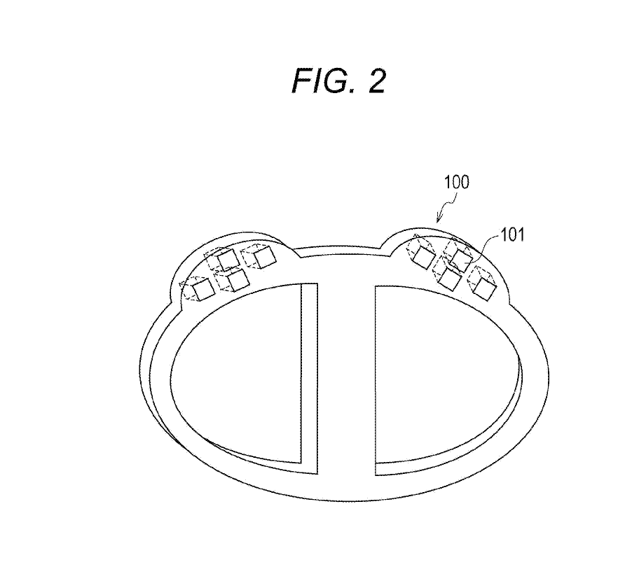

Next, using FIGS. 2 to 4, a specific configuration of a base phantom according to this embodiment is described. First, an exemplary base phantom for a breast examination suitable for the CT imaging is described by using FIG. 2.

A full-body CT apparatus images a CT image based on an image that is imaged when a radiation generating apparatus and a radiation detector are rotated centering on a body axis of an examinee. Accordingly, the base phantom has a structure simulating a range that is rendered when the breast examination is performed using the full-body CT apparatus. In a position of the breast, an insertion hole 101 is provided such that the evaluating phantom, described below, can be inserted (combined). Here, the base phantom is provided with a plurality of rectangular parallelepiped insertion holes 101; however, it is not limited to this as long as it is provided with at least one insertion hole 101. It is preferred that the insertion hole 101 be shaped such that a connecting phantom 300, described below, can be installed without any gap. Note that it is preferred that the gap be in a range not affecting the evaluation of the image that has been imaged. Furthermore, the insertion hole may also have a shape other than the rectangular parallelepiped; it may have any shape such as a cylindrical shape or a cubic shape as long as the connecting phantom can be installed. A base phantom 100, for example, has a thickness of 1 cm to 30 cm in a body axis direction. It is preferred that the base phantom 100 be constituted of a material having a radiation absorption value substantially equal to that of a structure of a human body. The base phantom 100 may also contain a structure corresponding to a heart, a lung, a mediastinum, a spine, and the like (not illustrated) other than the breast so as to simulate the structure within a human body. In this case, it is preferred that each of the structures be constituted of a material having the radiation absorption value substantially equal to that of the corresponding structure of the human body. The base phantom 100 may be constituted of a substance having a radiation absorption value similar to that of the human body as a whole or by combining substances having two to three types of the radiation absorption value so as to replace the structure of the human body with a simple structure. The base phantom 100 may also have a structure simulating a mammary gland structure, a fat tissue, and furthermore a vascular structure. As the material of the base phantom 100, for example, an acrylic container filled with water and a material such as urethane may preferably be used, but it is not to be limited to these.

The base phantom 100 may also be a combination of a plurality of divided base phantoms. For example, the base phantom 100 may be a combination of the base phantoms having different shapes representing a human body cut into round slices. In this case, by preparing and overlapping a plurality of the base phantoms, it is possible to extend and use it in the body axis direction. The plurality of divided base phantoms may also have a coupling portion coupling each of them or a fixing member fixing each of them. For example, the base phantom 100 may have a structure provided with a concave portion and a convex portion that are coupled together. In this case, when imaging by installing a plurality of base phantoms 100 on a full-body CT imaging table (not illustrated), it is possible to prevent each of them from being separated. It is also possible to fix the base phantoms 100 by using a rod-shaped fixing member (not illustrated) that fixes the plurality of divided base phantoms by penetrating therethrough. It is preferred that the rod-shaped fixing member be constituted of a substance having a radiation absorption value equal to that of the human body. The fixing member is constituted to be a part of the base phantom 100. As a different shape of the fixing member, a fixing member (not illustrated) that sandwiches and fixes the base phantom 100 may also be used.

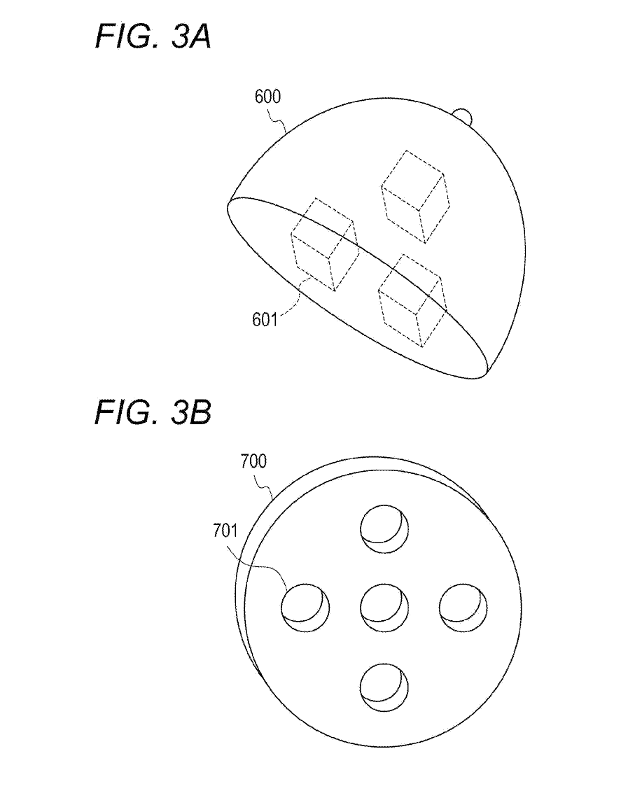

Next, a base phantom suitable for a radiographing apparatus and a tomosynthesis apparatus for the breast is described by using FIGS. 3A and 3B. FIG. 3A illustrates a base phantom 600 suitable for the radiographing apparatus for the breast and the radiation tomosynthesis apparatus for the breast.

FIG. 3A illustrates a structure simulating a range that is rendered by the radiographing apparatus and the radiation tomosynthesis apparatus for the breast. Since the breast is imaged in a pressed state, the apparatus has a structure enabling to easily press it in a state of being arranged in the apparatus. A plurality of rectangular parallelepiped insertion holes 601 is provided in a position corresponding to the breast such that the evaluating phantom can be inserted. The base phantom 600 has a thickness of 3 cm to 10 cm in the body axis direction. The base phantom 600 is constituted of a substance having a radiation absorption value similar to that of the human body. The base phantom 600 may also have a structure simulating a mammary gland structure, a fat tissue, and furthermore a vascular structure by combining substances having two to three types of the radiation absorption value. In the same way as the insertion hole 101 provided to the base phantom 100, the insertion hole 601 is shaped such that the connecting phantom 300 can be installed without any gap. Each evaluating phantom can be inserted into an insertion hole 301 provided to the connecting phantom 300.

FIG. 3B illustrates a structure simulating the breast in a pressed state. The radiographing apparatus and the radiation tomosynthesis apparatus for the breast image the breast in the pressed state, whereby imaging and evaluating becomes easy by shaping the evaluating phantom in a pressed shape in advance. An insertion hole 701 is provided such that the evaluating phantom is installed. It is preferred that the insertion hole 701 be shaped such that each of the evaluating phantoms, described below, can be installed without any gap. In the same way as the insertion hole 601, the insertion hole 701 is shaped such that the connecting phantom 300 can be installed without any gap. Note that it is preferred that the gap be in a range not affecting the evaluation of the image that has been imaged. A base phantom 700 may have a disk-shaped configuration as illustrated or may further have a structure such as a nipple as the configuration. By using the above-described base phantom, it is possible to evaluate lesion image extraction performance of the radiographing apparatus and the radiation tomosynthesis apparatus for the breast or the lesion image extraction performance of the imaging systems for both.

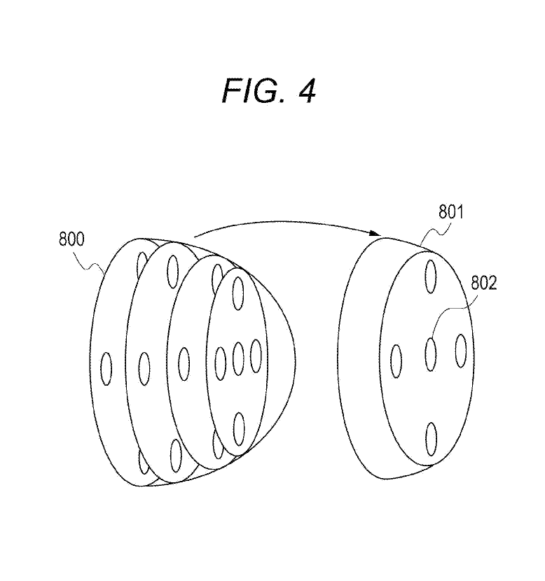

FIG. 4 is a base phantom 800 suitable for a breast specific CT apparatus. The base phantom 800 is constituted of a plurality of disk-shaped base phantoms 801. By combining each of them, a structure simulating a shape of the breast is formed. The breast specific CT apparatus images the breast in a state of being distorted by its own weight or in a state of being fixed by a fixing member. The disk-shaped base phantom 801 may also have a structure simulating a mammary gland structure, a fat tissue, and furthermore a vascular structure by combining a plurality of materials each having a different radiation absorption value. Since the base phantoms 800 can be combined with each other, the shape of the breast of a desired size can be constituted. Each of the disk-shaped base phantoms 801 may have a structure provided with a coupling portion such as of a concave and convex. It is also possible to use a rod-shaped fixing member (not illustrated) that fixes the plurality of disk-shaped base phantoms 801 by penetrating therethrough. It is preferred that the rod-shaped fixing member be constituted of a substance having a radiation absorption value equal to that of the human body, and the rod-shaped fixing member constitutes a part of the base phantom 800 for a breast examination by the breast specific CT apparatus. It is also possible to use the fixing member (not illustrated) that fixes the plurality of disk-shaped base phantoms 801 by aligning the plurality of disk-shaped base phantoms 801 and by sandwiching it from outside. In the same way as the above-described base phantom, the disk-shaped base phantom 801 is provided with a plurality of insertion holes 802 such that each of the evaluating phantoms can be inserted therein.

By using the base phantom as above, it is possible to perform the imaging using the breast specific CT apparatus and to evaluate the lesion image extraction performance of the apparatus.

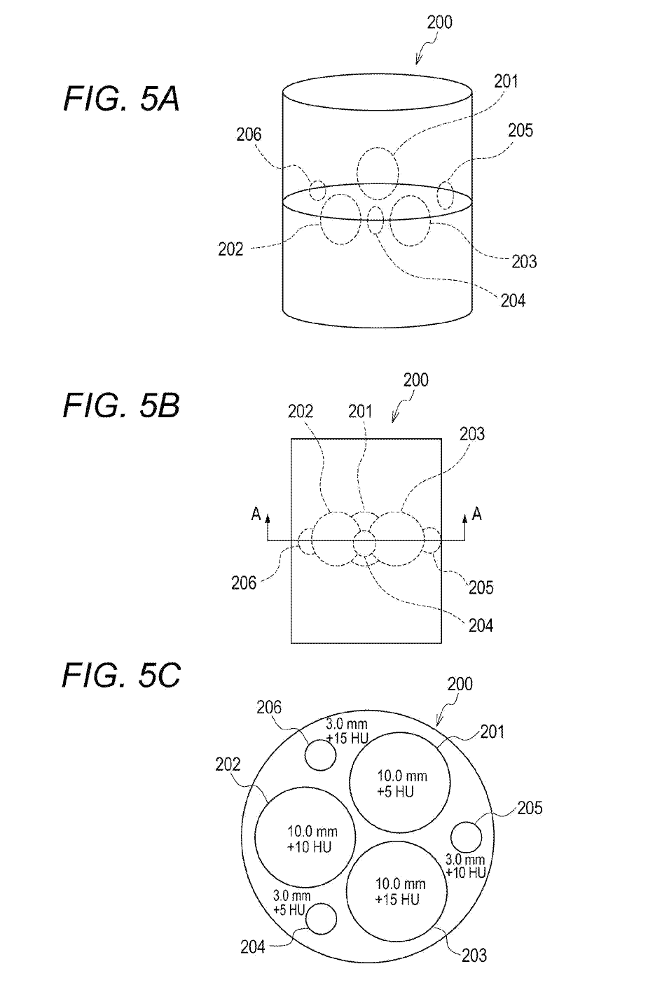

Next, the evaluating phantom is described in detail by using FIGS. 5 to 7. Each of the evaluating phantoms has an index (evaluation region) corresponding to at least one evaluation among a low contrast resolution, a high contrast resolution, and a spicule resolution.

Using FIGS. 5A, 5B, and 5C, a low contrast resolution evaluating phantom is described. FIG. 5A is a perspective view of the low contrast resolution evaluating phantom. FIG. 5B is a front view of the low contrast resolution evaluating phantom, and FIG. 5C is a sectional view taken along direction A-A of FIG. 5B.

An overall structure of a low contrast resolution evaluating phantom 200 excluding indexes (201 to 206 in FIGS. 5A to 5C) is configured to include a substance having a radiation absorption value equal to that of the base phantom 100. In particular, the low contrast resolution evaluating phantom is used for evaluating detection performance of a large index having a small contrast difference (Hounsfield unit value difference) with a surrounding part.

Inside the low contrast resolution evaluating phantom 200, as the indexes (201 to 206 in FIGS. 5A, 5B, and 5C), spherical or cylindrical structures each simulating a tumor lesion, which is characteristic of a breast cancer, are aligned and enclosed in a predetermined arrangement by a size and by a difference in the radiation absorption value. The low contrast resolution evaluating phantom 200, as an exemplary structure, has a cylindrical structure having a bottom face diameter of 2 cm to 3 cm and a height of 2 cm to 5 cm. Here, the indexes 201 to 206 are spherical substances, each having a diameter of 2 mm to 10 mm, and are constituted of substances each having a radiation absorption value varied between −100 and 100 of the Hounsfield unit value (HU value) compared to the evaluating phantom 200 as a whole. Here, a whole part of the low contrast resolution evaluating phantom 200 excluding the indexes may be constituted of, for example, hydroxyapatite, urethane, and the like. By adjusting a hydroxyapatite component, it is possible to set the HU value of an index part of the low contrast resolution evaluating phantom 200.

The low contrast resolution evaluating phantom 200 may also have a structure simulating a mammary gland structure, a fat tissue, and furthermore a vascular structure. In a case where all of the substances for the evaluation cannot be contained in one evaluating phantom, the low contrast resolution evaluating phantom 200 may be used divided into more than one phantom.

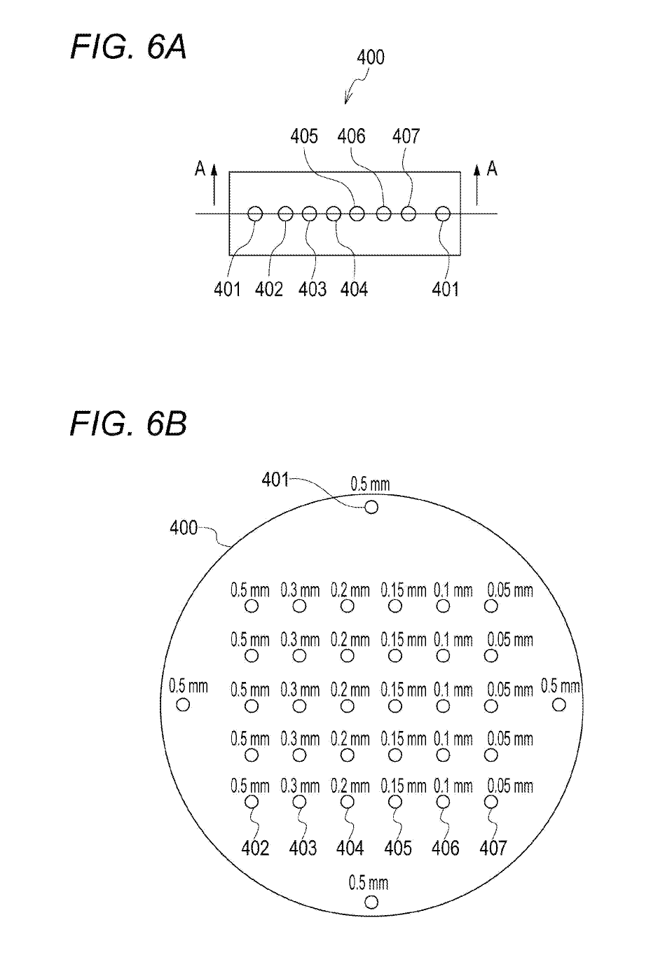

The high contrast resolution evaluating phantom is described using FIGS. 6A and 6B. FIG. 6A is a front view of the high contrast resolution evaluating phantom, and FIG. 6B is a sectional view of the high contrast resolution evaluating phantom taken along direction A-A of the front view.

A high contrast resolution evaluating phantom 400 is constituted of at least a metal piece 401 for checking a position and indexes 402 to 407 simulating microcalcification. The high contrast resolution evaluating phantom 400 has a cylindrical structure and a bottom face diameter of 2 cm to 3 cm and a height of 1 cm to 5 cm as the phantom as a whole.

The indexes 402 to 407 are metal pieces simulating the microcalcification, which is characteristic of the breast cancer, each having a different size and a different shape. Each of the indexes is arranged to a predetermined position within the high contrast resolution evaluating phantom 400. These metal pieces, for example, are constituted of an aluminum piece, hydroxyapatite, and the like. The high contrast resolution evaluating phantom 400 as a whole is constituted of a substance having a radiation absorption value equal to that of the base phantom 100. The high contrast resolution evaluating phantom 400 as a whole, for example, may be constituted of a mixture of the hydroxyapatite, oil, and urethane in the same way as the low contrast resolution evaluating phantom 200 as a whole.

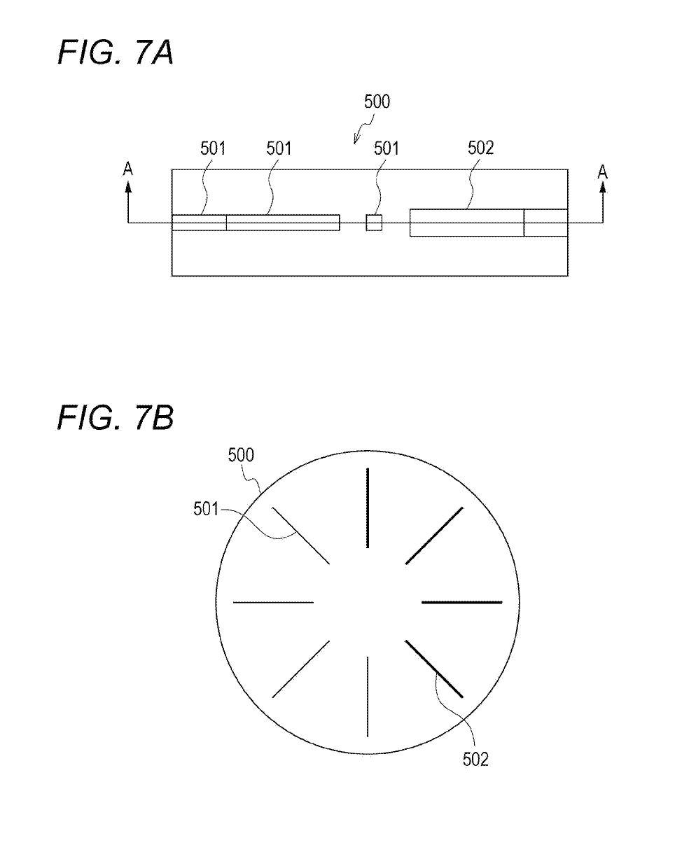

A spicule resolution evaluating phantom is described using FIGS. 7A and 7B. FIG. 7A is a front view of the spicule resolution evaluating phantom. FIG. 7B is a sectional view of the spicule resolution evaluating phantom taken along direction A-A of the front view. A spicule resolution evaluating phantom 500 is configured to include at least indexes 501 and 502. Here, a spicule refers to a shape in which the mammary gland structure is retracted, which is characteristic of the breast cancer. The spicule resolution evaluating phantom 500 is shaped, for example, in a cylindrical structure having a bottom face diameter of 2 cm to 3 cm and a height of 1 cm to 5 cm. The spicule resolution evaluating phantom 500 encloses the plurality of indexes 501 and 502, each simulating the spicule, radially arranged by the thickness and by the size. For example, the indexes 501 and 502 are constituted of nylon and the like. In this embodiment, two types of the indexes are illustrated; however, it is not to be limited to two types, and more indexes may be provided as well. Since each of the evaluation regions of the evaluating phantom is attachable to and detachable from the base phantom and can be shared, it is possible to reduce variation in evaluation caused by a manufacturing error of the evaluation region.

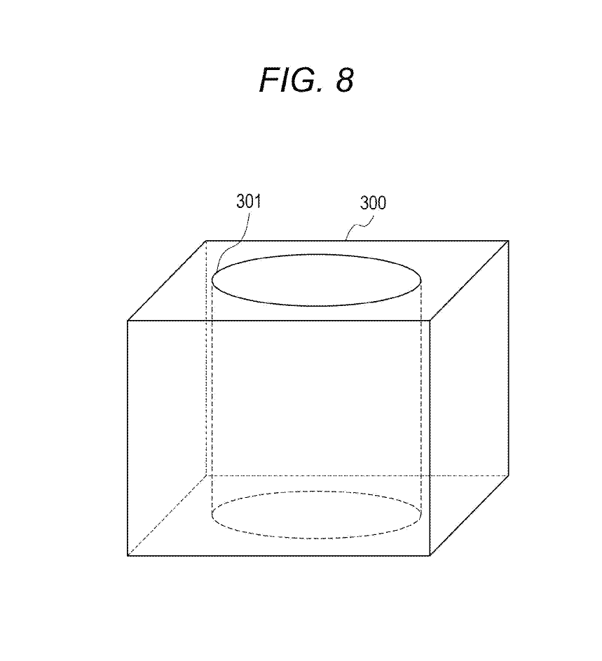

A connecting phantom is described using FIG. 8. FIG. 8 is a perspective view of a connecting phantom 300. The connecting phantom 300 is a member for arranging the evaluating phantom to the base phantom. The connecting phantom 300 is configured to be a size allowing it to be arranged in an insertion hole provided to the base phantom. The connecting phantom 300 is provided with an insertion hole 301 in which the evaluating phantom is inserted. The insertion hole 301 is configured to be a size allowing each of the evaluating phantoms to be inserted therein. Then, a shape of an outer periphery of the connecting phantom 300 is substantially the same as the shape of the insertion hole 101 of the base phantom 100. A material constituting the connecting phantom 300 is a substance having a radiation absorption value equal to that of the base phantom 100 that is to be inserted. By using the connecting phantom 300, it is possible to stably arrange the evaluating phantom inside the insertion hole of the base phantom.

A case in which the connecting phantom is arranged to each of the base phantoms is described. The connecting phantom 300 having the evaluating phantom, which is inserted in the insertion hole 101 of the base phantom 100, is arranged. Since the connecting phantom 300 is a rectangular parallelepiped, it is possible to select from a plurality of axial directions when arranging. Accordingly, by imaging in an arbitrary direction, it is possible to evaluate an influence of an arrangement direction on the image. It is also possible to shape the insertion hole 101 of the base phantom 100 to be a regular icosahedron or a sphere and to use an adapter having the same shape. Accordingly, the arrangement direction of the evaluating phantom becomes more flexible, whereby it is possible to perform the evaluation by using many arrangement angles. In a case where a gap exists due to the height of each of the evaluating phantoms when it is arranged inside the insertion hole 301 of the connecting phantom 300, the gap may be filled by using a spacer phantom (not illustrated) having the same bottom face as the evaluating phantom and a structure with an appropriate height.

As above, it is possible to arrange the evaluating phantom to the selected base phantom by using the connecting phantom 300, whereby the lesion image extraction performance of each of the imaging systems can be evaluated.

Next, there is described an evaluation method of an image using an image of the evaluating phantom that is imaged.

There is described a procedure of comparing the lesion image extraction performance based on an image in which the evaluating phantom is imaged by using each of the imaging systems. Here, there is described, for example, a case in which the imaging system suitable for evaluating the low contrast resolution is determined. The indexes 201 to 206 of the low contrast resolution evaluating phantom 200, which is installed in the base phantom, imaged by using each of the imaging systems are displayed on a monitor. From among the displayed images, an engineer can select an image in which the indexes are more preferably distinguishable. As above, by comparing capability to be extracted in each examination of the index 206 that is imaged by using each of the imaging systems, it is possible to determine an imaging system optimal for evaluating a specific lesion, a shape thereof, a size thereof, and the like. Note that the evaluation method of the image is not to be limited to this, and it is possible to automatically select an imaging system optimal for the evaluation index by quantitatively analyzing image data. In the same way, it is possible to determine an imaging system optimal for detecting a lesion simulated by each of the high contrast resolution evaluating phantom 400 and the spicule resolution evaluating phantom 500.

### Second Embodiment

There is described a calibration method of a radiographing apparatus according to a second embodiment. In the first embodiment, based on an image obtained as a result of imaging an evaluating phantom, performance evaluation among imaging systems has been performed. On the other hand, in this embodiment, based on an image in which the evaluating phantom is imaged, calibration of lesion image extraction performance is performed. The calibration method includes a selection step, a combining step, and a calibration step. The selection step and the combining step are the same as those in the first embodiment. In the calibration step, based on the image obtained as a result of imaging the evaluating phantom, the calibration of the lesion image extraction performance by the selected imaging system is performed. Accordingly, it is possible to perform the calibration by using a uniform index among the radiographing apparatuses, each having a different imaging system. By using the above calibration method, compared to a case in which a phantom specialized for each of the imaging systems is used, it is possible to reduce a difference between images in which the same specimen is imaged. By using this calibration method, it is also possible to compare intensity and radiation quality of radiation to be irradiated for providing substantially equal lesion image extraction performance among the imaging systems. By this calibration method, it is possible to determine the intensity and the radiation quality of the radiation. Accordingly, an examinee, an engineer, and the like can select an imaging system with a small radiation dosage or an imaging system with a short imaging time from among different imaging systems.

Note that the embodiments of the present invention may also be achieved by a computer or a control computer executing a program (computer program). Also, a unit for supplying the program to the computer, for example, a computer-readable recording medium such as a CD-ROM recording such program and a transmission medium such as the Internet transmitting such program, is also applicable as an exemplary embodiment of the present invention. Furthermore, the above-described program is also applicable as an exemplary embodiment of the present invention. The above-described program, the recording medium, the transmission medium, and a program product are within a scope of the present invention. An embodiment of the invention that is a combination easily imaginable from the embodiments is also within the scope of the present invention.

According to the present invention, it is possible to provide an evaluation method for a radiographing apparatus by which an imaging system suitable for a target evaluation region can be selected from among a plurality of imaging systems.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2014-167821, filed Aug. 20, 2014, which is hereby incorporated by reference herein in its entirety.

What is claimed is:

1. An evaluation method for a radiographing apparatus, the evaluation method comprising:
an imaging step of imaging a plurality of base phantoms, each of the base phantoms corresponding to a different imaging system and combined with a common evaluation region; and
an evaluation step of performing evaluation among the different imaging systems based on an image in which the common evaluation region has been imaged in the imaging step.

2. The evaluation method for the radiographing apparatus according to claim 1, the evaluation method further comprising
a combining step of combining a common evaluating phantom having the evaluation region in each of the plurality of base phantoms.

3. The evaluation method for the radiographing apparatus according to claim 1, wherein
the imaging system includes at least one imaging system among CT imaging, tomosynthesis imaging, and two-dimensional imaging.

4. The evaluation method for the radiographing apparatus according to claim 1, wherein
the evaluation region has an index corresponding to at least one evaluation among low contrast resolution, high contrast resolution, and spicule resolution.

5. The evaluation method for the radiographing apparatus according to claim 1, wherein
each of the base phantoms has a shape corresponding to at least one imaging system among CT imaging, tomosynthesis imaging, and two-dimensional imaging.

6. The evaluation method for the radiographing apparatus according to claim 2, the evaluation method further comprising:
a second combining step of combining a connecting phantom configured to combine the evaluating phantom with each of the base phantoms.

7. The evaluation method for the radiographing apparatus according to claim 6, wherein
the connecting phantom is arranged to each of the plurality of base phantoms in a direction corresponding to the imaging system.

8. The evaluation method according to claim 1, further comprising:
a selection step of selecting, based on the image that has been imaged in the evaluation step, one imaging system or one imaging apparatus suitable for imaging a lesion corresponding to the common evaluation region is selected from the plurality of imaging systems.

9. The evaluation method for the radiographing apparatus according to claim 1, wherein
based on the image that has been imaged in the evaluation step, intensity or radiation quality of radiation is compared between the different imaging systems when the different imaging systems have substantially equal lesion image extraction performance.

10. A radiographing apparatus for obtaining a radiographic image of a breast, wherein
imaging of the breast is performed by setting an imaging condition based on a result of evaluating the breast by using the evaluation method for the radiographing apparatus, the evaluation method comprising:
an imaging step of imaging a plurality of base phantoms, each of the base phantoms corresponding to a different imaging system and combined with a common evaluation region; and
an evaluation step of performing evaluation among the different imaging systems based on an image in which the common evaluation region has been imaged in the imaging step.

11. A phantom for evaluating a plurality of imaging systems, the phantom comprising:
a plurality of base phantoms, each of the base phantoms corresponding to a different imaging system; and
an evaluating phantom configured to be attachable to and detachable from each of the plurality of base phantoms, the evaluating phantom having an evaluation region for evaluating imaging performance of a radiographing apparatus.

12. The phantom according to claim 11, further comprising
a connecting phantom configured to combine the evaluating phantom with the plurality of base phantoms in a direction according to an imaging system corresponding to each of the plurality of base phantoms.

13. A calibration method for a radiographing apparatus, the calibration method comprising:
an imaging step of imaging a plurality of base phantoms, each of the base phantoms corresponding to a different imaging system and combined with a common evaluation region; and
a calibration step of performing, based on an image in which the common evaluation region has been imaged in the imaging step, calibration of lesion image extraction performance among the different imaging systems.
